(19) United States
(12) Patent Application Publication
(10) Pub. No.: US 2014/0199772 A1
(43) Pub. Date: Jul. 17, 2014

(54) PROCEDURE AND AUTOMATIC APPARATUS FOR IN VITRO BLOOD COAGULATION DIAGNOSTIC TESTS

(71) Applicant: DIAGON Kft., Budapest (HU)

(72) Inventors: Levente Kiss, Biatorbagy (HU); Kornel Nagy, Biatorbagy (HU); Matyas Petoe, Budapest (HU); Laszlo Izsak, Budapest (HU); Jozsef Antal, Budapest (HU)

(73) Assignee: DIAGON Kft., Budapest (HU)

(21) Appl. No.: 14/213,867

(22) Filed: Mar. 14, 2014

Related U.S. Application Data
(63) Continuation-in-part of application No. PCT/HU2012/000048, filed on Jun. 13, 2012.
(60) Provisional application No. 61/793,232, filed on Mar. 15, 2013.

(30) Foreign Application Priority Data
Nov. 16, 2011 (HU) P1100631

(51) Int. Cl. G01N 33/86

(52) U.S. Cl. CPC G01N 33/86
USPC 436/69; 422/63

(57) ABSTRACT

Apparatus and processes for automated diagnostic testing may include cuvette dispensing, test sample dispensing, reagent dispensing, incubation, photo-optical measuring, and a control in operative communication with the cuvette dispensing unit, the test sample dispenser, the reagent dispenser, the incubation unit, and the measuring unit. A cuvette removal location, a reagent removal location, a measuring location, and a collection receptacle may be situated along a first common circular arc having first geometric center. A cuvette-moving arm and a reagent-dispensing arm have a common axis of rotation passing through this geometric center. A sampling position, a sample dispensing position, an additional rinsing position, and an additional washing position, may all be provided on a second circular arc having second geometric center that has a second axis of rotation for a rotatable sampling arm of the test sample dispenser. The apparatus and processes may be employed for photo-optical analysis of blood plasma samples.

This application claims benefit as a non-provisional of copending U.S. provisional appl. No. 61/793,232 filed on Mar. 15, 2013, and the present application claims benefit as a C-I-P continuation-in-part of copending PCT International application no. PCT/HU2012/000048 filed on Jun. 13, 2012 designating the U.S., claiming benefit of priority to prior Hungarian national application no. HU-P1100631 filed on Nov. 16, 2011, this priority claim being identically applicable to the present application, and U.S. provisional appl. Ser. No. 61/793,232 as well as parent PCT appl. no. PCT/HU2012/000048 are entirely incorporated herein by reference in their entireties and as to all their parts, for all intents and purposes, as if identically set forth in full herein.

The present disclosure relates to procedures and automatic apparatus for in vitro blood coagulation diagnostic tests. Solutions according to the present disclosure may achieve a compact, fast and user-friendly method for performing a large number of diagnostic tests.

In the present disclosure a sample tube is a cylindrical or prism-shaped vessel containing the test sample, mostly made of glass or transparent plastic, dosed at one end and lockable at the other end, which may be, for example, a tube or vial. The test sample may be a liquid or solid organic or inorganic material.

The sample container racks according to the present disclosure are used for placing and storing sample tube/tubes described above.

In the present disclosure a reaction mixture is a mixture of the test reagent/reagents and the test sample/samples participating in the examined reaction and determining the examined reaction.

In the present disclosure a cuvette is an optically transparent cylinder or prism favorably made of glass or plastic, which is closed at the bottom and open at the top and can have various different appearances both in respect of its geometry and size, which functions as the reaction space and storage place of the test sample or reaction mixture according to the invention.

Similarly to different automatic bioanalytical instruments, in in vitro blood coagulation diagnostic tests (for example plasma PT prothrombin time determination, APTT activated partial thromboplastin time determination, etc.) also, the construction of automatic apparatuses is basically determined by expectations set by the large number of tests. A general characteristic feature of these automatic apparatuses is that they have functions such as dispensing cuvettes, dispensing test samples, dispensing test reagents, incubating test sample and/or reaction mixture, measuring reaction desired in the reaction mixture and removing used cuvettes. These functions are carried out under controlled temperature circumstances, and the consecutive order of the functions is ensured with a large number of partly or completely electronically controlled forwarding and dispensing structures installed along the locations carrying out the functions.

In the apparatus described in published PCT Intn'l. appl. no. WO 93/15408A1, sample holders that can be identified with a barcode are placed on a rotatable disc having a sensing and moving system, using a moving unit designed for this purpose. The sample holders are filled with reagent dispenser and sample dispenser structures installed along the disc. In a preferred position of the disc the sample holders filled with the reaction mixture are heated. In further preferred positions of the disc, in the case of a photodetector suiting the test type, the reaction according to the test is measured. After measuring, the sample holders are removed from the disc using a moving structure designed for this purpose. The advantage of the apparatus is that the sample holders are easily accessible on the rotatable disc. From the aspect of saving space and time, it is less favorable to place the dispensing functions on structures moved on separate axles. A further disadvantage derives from the advantage of the construction: by performing the incubation, measuring and forwarding functions on the same disc, the rate of the different reactions slows down.

U.S. Pat. No. 5,439,646 describes an apparatus in which measuring modules are placed along the edge of a disc rotating in two directions, with cells inside them accommodating test samples and reagents. In all measuring modules LED lights and photodetectors are used for measuring the given reactions. The cells are inserted, filled with sample/samples and reagent/reagents, and removed after measuring, by separately mounted arms. The advantage of the construction is that different coagulation tests can be measured simultaneously. The disadvantage of the construction is that it is space demanding and complicated.

U.S. Pat. No. 7,916,298B2 provides a solution for increasing efficiency in in vitro blood coagulation diagnostic tests by duplicating the apparatus. However, this performance increasing procedure increases the possible number of technical errors. A further disadvantage of the construction of the device is that its large space demand restricts wide laboratory use. The space- and time-demanding operation of the dispenser structures mounted on several separate axles is also a disadvantage.

In the apparatuses described in U.S. Pat. Nos. 7,931,861B2 and 7,931,863B2, a more favorable access to the individual operations is ensured with the concentric arrangement of cuvettes on rotatable discs. The disadvantages, of the constructions are that the dispensing of reagents, the dispensing of samples, and the movement of the cuvettes is solved with arms mounted on separate axles. A further disadvantage of that solution is that there is no preheating of the cuvettes forwarded from the cuvette dispenser to the rotatable disc.

In the apparatus described in U.S. Pat. No. 7,962,292B2, a cuvette dispenser places the cuvettes on a disc rotating in two directions, a preheated test reagent is dispensed in the cuvettes with arms moved on different axles, and then the cuvettes filled with the reaction mixture to be measured are forwarded to heated measuring points. The advantage of the construction of the apparatus is that the concentric arrangement of the cuvettes on the rotatable discs ensures a more favorable accessibility of the individual operations. However, from the aspects of space and time demand it is less favorable to carry out a given function with dispensing arms mounted on several different axles (for example reagent-dispensing arms). A further disadvantage is that there is no preheating of the cuvettes forwarded from the cuvette dispenser to the rotatable disc.

In U.S. Pat. No. 7,955,556B2 the automatic apparatus was constructed to suit urgent sample analyses interrupting the routine work process. Besides the routine sample dispenser the apparatus also has an emergency sample dispenser and measuring point. The significant space demand of the duplicate system, that was originally constructed for urine tests but is also adaptable for other bioanalytical tests, may only be fulfilled in special laboratories. A further disadvantage of the duplicate system derives from its advantage: when urgent sample analysis is performed, the routine measuring process is suspended, and it can only be continued after finishing the urgent sample analysis.

The present disclosure elaborates solutions for in vitro blood coagulation diagnostic tests, with which the performance of various tasks may be simplified, has a space-saving compact construction, is fast in operation and is user-friendly by being easy to handle. It is advantageous to create a solution with an integrated construction, which, due to its construction and operation, makes it unnecessary to use additional moving structures.

In order to save space, practically, an integrated modular construction should be created for performing the functions described in detail in the introductory part, a further advantage of which is that, with its use, the successive continuity of various functions may be ensured without additional moving structures. In accordance with this, favorably created modular construction has cuvette-dispenser module, incubation module, module moving sample container racks, emergency module, reagent holder module, measuring module and sampling, cuvette-moving and reagent-dispensing arms serving the above modules.

Advantageously, the cuvette removal location of the incubation module, the reagent removal location of the reagent holder module and the measuring locations of the measuring module are arranged along a circular arc, and placement of the sample cuvettes at the measuring points, dispensing of the reagent in the cuvettes placed in the measuring module; and, after finishing measurements, forwarding of used cuvettes to the receptacle are realized with cuvette-moving and reagent-dispensing arms moved in different planes from the common geometric center of the circular arc created according to the above as from a common center of rotation, with a common axis of rotation. Practically, rinsing and washing positions are created for the reagent-dispensing arm. By employing solutions according to the invention, a significant number of additional moving structures may be spared. On the basis of experiments, further advantages derive from a further favorable solution, according to which further similarly preferred positions are created in the case of further procedural steps, where sampling position, sample dispensing position, and further rinsing and washing positions are created along a further circular arc, and sampling arm is operated in the geometric center of this further circular arc on a further axis of rotation.

The present disclosure relates to a procedure for in vitro blood coagulation diagnostic tests, in the course of which favorably a series of tests is performed, where empty cuvettes are kept under controlled temperature circumstances, test samples placed in sample tubes arranged in sample container racks are forwarded to the cuvettes, and, if necessary, test reagent (reagents) is (are) dispensed in the cuvettes. The cuvettes with the test samples and, if necessary, reagent(s) inside them are incubated for a desired period of time, and blood coagulation reactions of the test samples are measured according to photo-optical principles. Then the cuvettes are removed, and the individual steps and their order are attuned and automated with the help of a control unit, favorably a computer. The procedure is based on that, during the tests, incubated cuvette removal point, test reagent removal point and test sample measuring point are handled as preferred positions. These preferred positions are arranged along the same circular arc, and in the geometric center of this circular arc, on a common axis of rotation, cuvette-moving arm and reagent-dispensing arm are operated. Rinsing and washing positions are created for the reagent-dispensing arm, and a receptacle is created for removing the cuvettes after measuring. At the same time, in the case of further procedural steps, further similarly preferred positions are created, where sampling position, sample dispensing position and further rinsing and washing positions are created along a further circular arc. In the geometric center of this further circular arc, on a further axis of rotation sampling arm is operated, and the cuvette-moving arm, the reagent-dispensing arm and the sampling arm are moved along the circular arc and the further circular arc in horizontal and vertical planes.

In a favorable realization of the procedure, a further preferred position is created along a further circular arc to function as an emergency disc removal point.

In a further favorable form of realization of the procedure according to the invention the cuvette-moving arm and the reagent-dispensing arm operated on a common axis of rotation are moved in different planes.

Favorably, the cuvette-moving arm and the reagent-dispensing arm moved on a common axis of rotation in different planes are operated in such a manner that the movement of the reagent-dispensing arm in the vertical plane is locked by the movement of the cuvette-dispensing arm in the horizontal plane.

The sampling arm is operated in such a way that in the sampling position of the sampling arm the movement of the sample container rack is locked; in the sample dispensing position of the sampling arm the movement of the emergency disc and the movement of the incubation disc is locked in sample dispensing position.

In a further favorable form of realization of the procedure, in the reagent-dispensing position of the reagent-dispensing arm the movement of the incubation disc is locked in reagent-dispensing position, and the movement of the reagent holder disc is locked in reagent removal position.

The present disclosure also relates to an automatic apparatus for in vitro blood coagulation diagnostic tests, favorably for the realization of the procedure described above, which apparatus has a cuvette-dispensing unit, a test sample dispenser and a reagent dispenser. Furthermore, it contains an incubation unit and a measuring unit for the photo-optical measurement of blood coagulation reactions of blood plasma samples, and it also has a control unit, favorably a computer, connected to these. The automatic apparatus is constructed in such manner that its test sample dispenser contains a module moving the sample container racks and a sampling arm. Its reagent dispenser has a reagent holder module and a reagent-dispensing arm. Its further units have a modular construction, where the output of the cuvette-dispensing module joins a system of storage tracks connected to the nests of the rotatable disc of the incubation module. The cuvette removal point of the incubation module, the reagent removal point of the reagent holder module, the measuring point of the measuring module and the receptacle where the cuvettes are collected after measuring are all arranged along the same circular arc. In the geometric center of the circular arc there is the common axis of rotation of the cuvette-moving arm and the reagent-dispensing arm moved in horizontal and vertical planes. The reagent-dispensing arm has rinsing and washing positions. Furthermore, the sampling position of the module moving the sample container racks, one cuvette nest of the rotatable disc of the incubation module, the removal point of a further emergency module, and further rinsing and washing positions of the sampling tip are arranged along a further circular arc. The axis of rotation of the sampling arm is placed in the geometric center of the further circular arc.

According to the present disclosure, the cuvette-moving and reagent-dispensing arms mounted on the common axis of rotation in a movable way, and the sampling arm positioned beside the module moving the sample container racks may be favorably moved by electronically controlled electric motors, or by electronically controlled hydraulic or pneumatic drives. The harmonic co-action of the cuvette-moving and reagent-dispensing arms mounted on the common axis of rotation in a movable way is ensured in such a way that the vertical movement of the reagent-dispensing arm is locked by the horizontal movement of the cuvette-dispensing arm.

An exemplary form of execution of the automatic apparatus, is described in detail on the basis of the attached drawings, where:

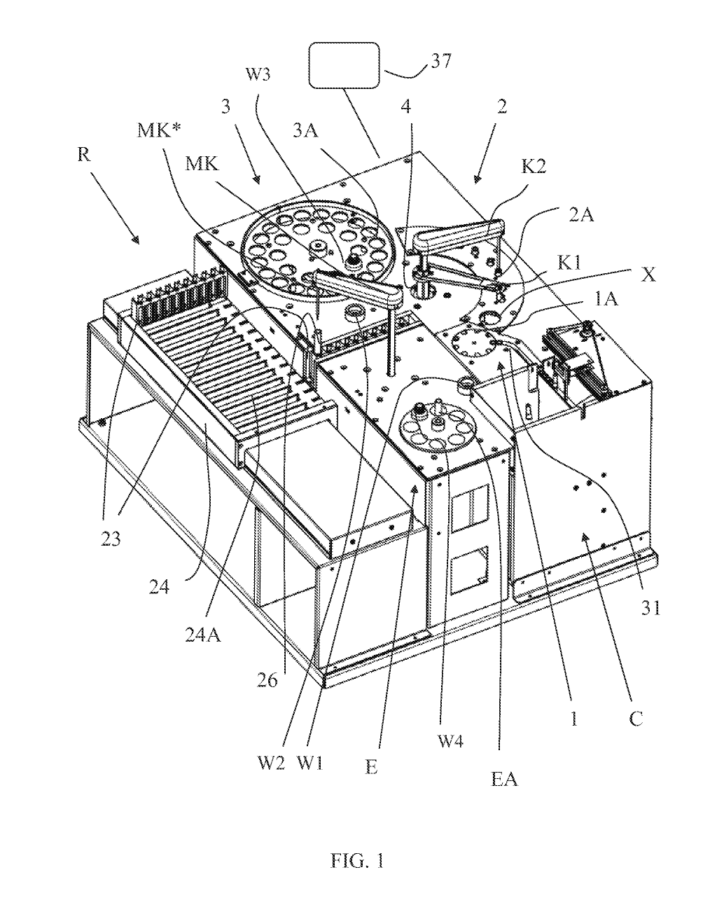

FIG. 1 shows a view of the apparatus,

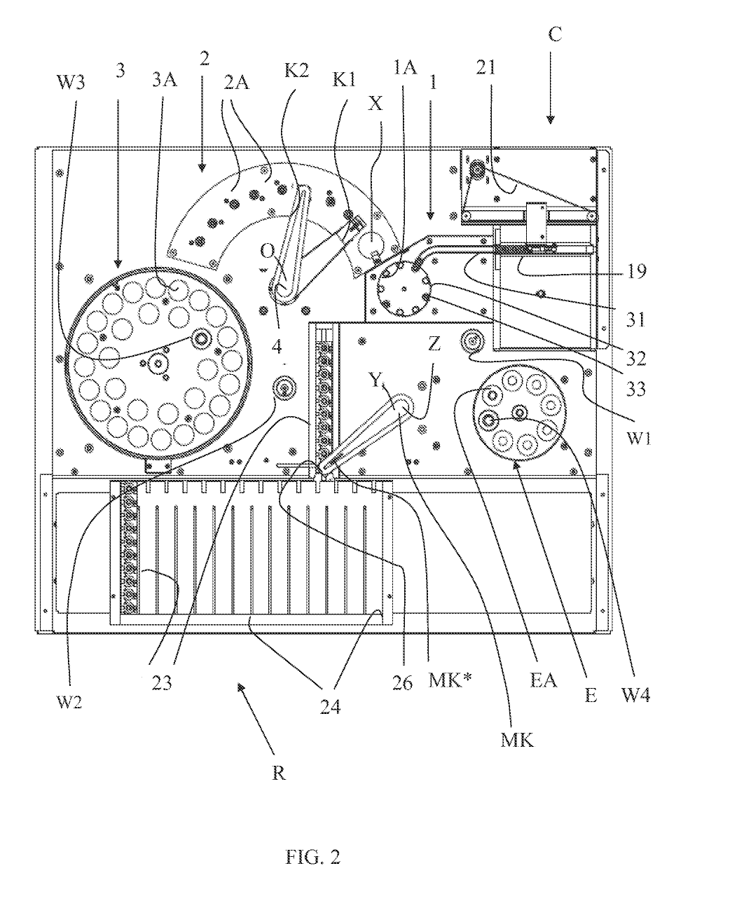

FIG. 2 shows the top view of the apparatus,

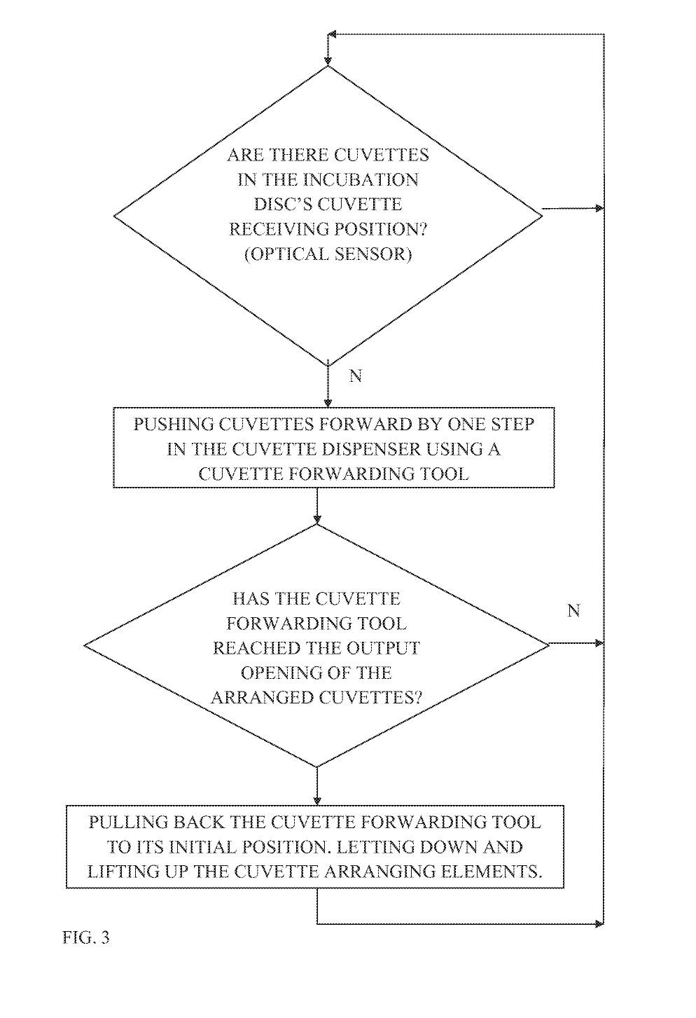

FIG. 3 shows the flowchart of the operation of the cuvette dispenser,

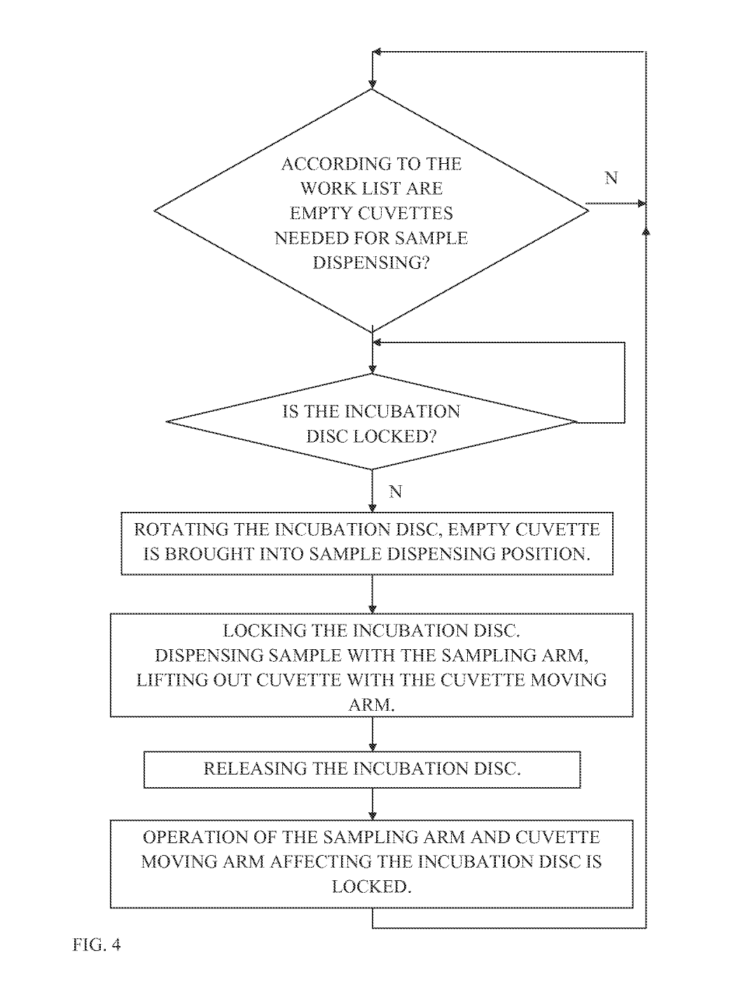

FIG. 4 shows the flowchart of the operation of the incubation module,

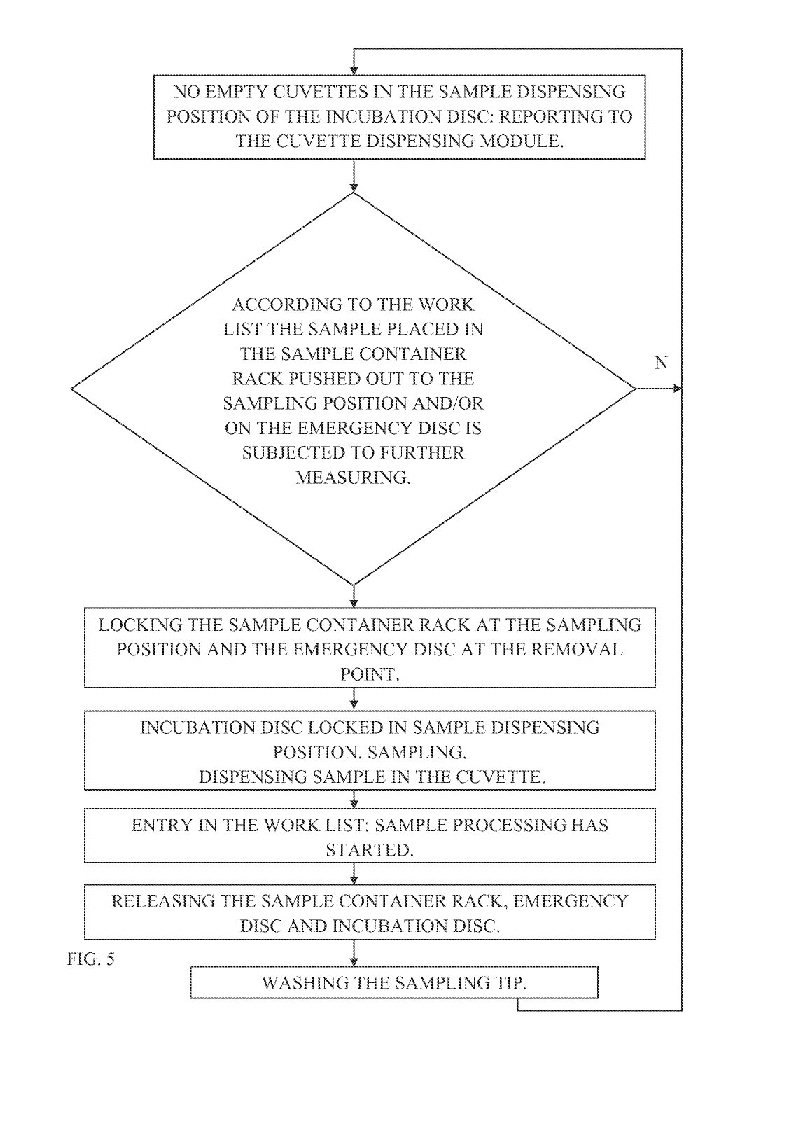

FIG. 5 shows the flowchart of the operation of the sampling arm,

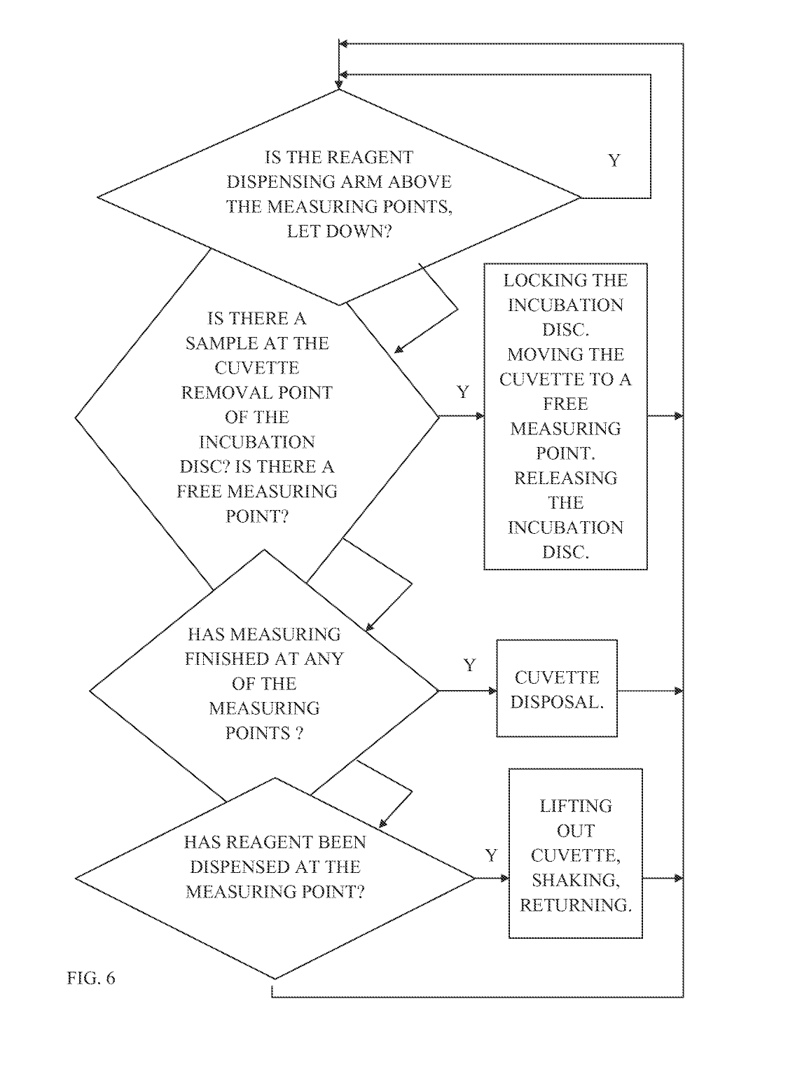

FIG. 6 shows the flowchart of the operation of the cuvette-moving arm,

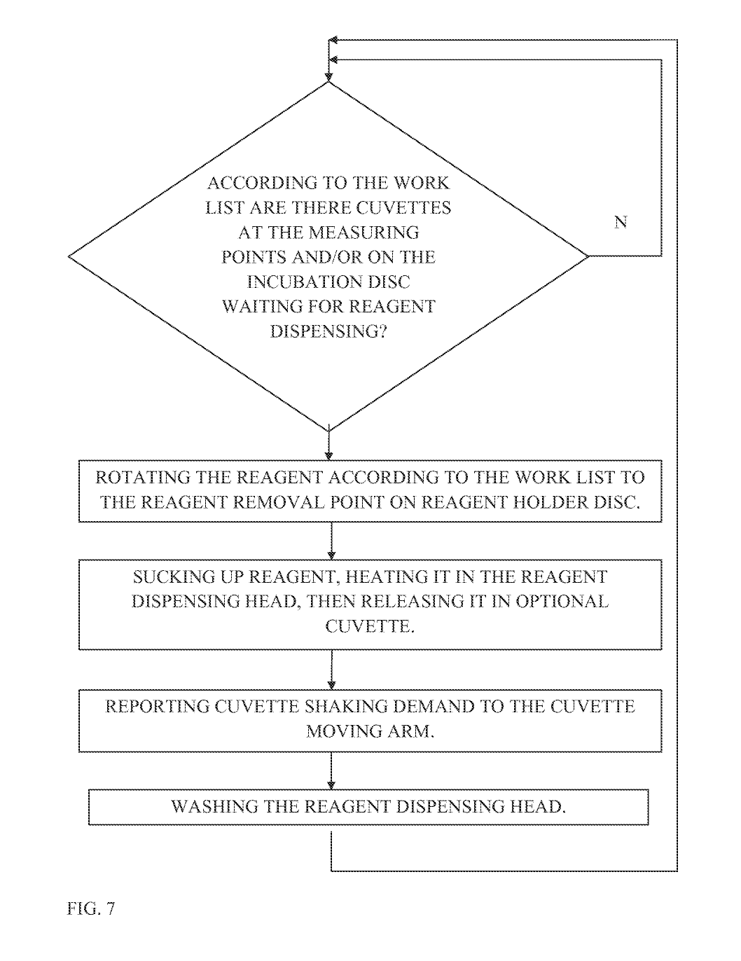

FIG. 7 shows the flowchart of the operation of the reagent holder module and the reagent-dispensing arm, and

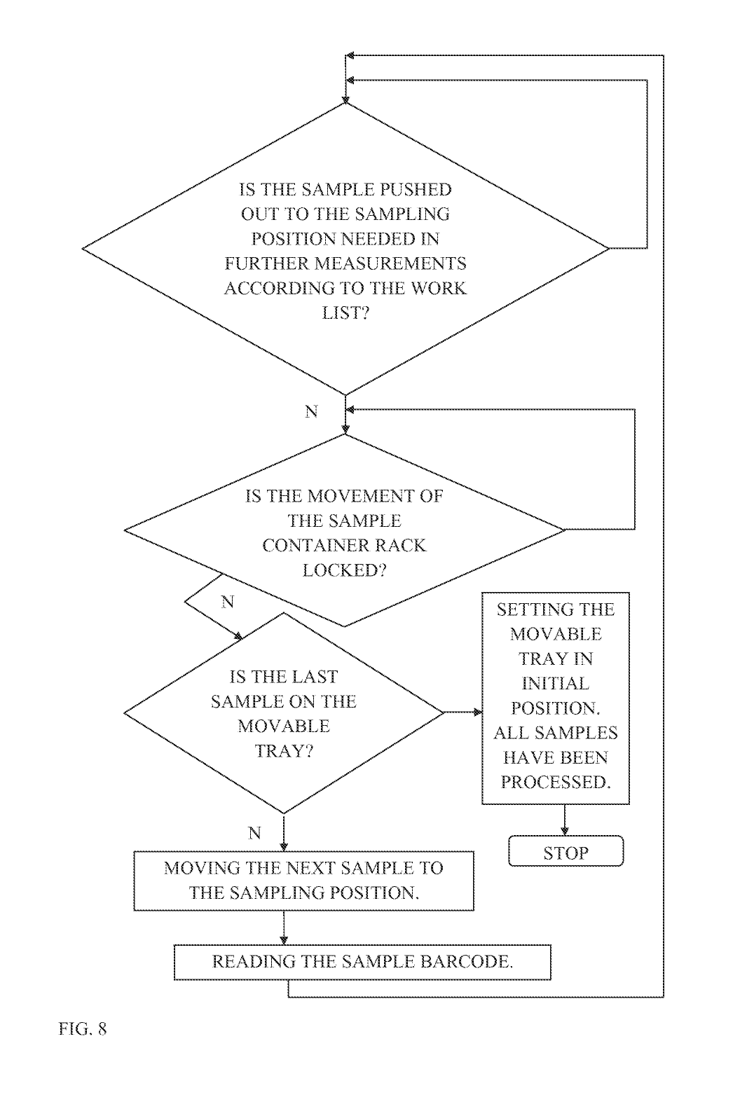

FIG. 8 shows the flowchart of the operation of the module moving the sample container racks.

In the following description, numerous specific details are set forth in order to provide a thorough understanding of versions of the present invention. It will be apparent, however, to one skilled in the art that versions of the present invention may be practiced without some of these specific details. Furthermore, as used throughout this specification, the terms ‘a’, ‘an’, ‘at least’ do not denote a limitation of quantity, but rather denote the presence of at least one of the referenced item, and the term ‘a plurality’ denotes the presence of more than one referenced items.

FIG. 1 shows a view of the automatic apparatus according to the invention, which has a cuvette-dispensing unit, a test sample dispenser and a reagent dispenser, and it contains an incubation unit and a measuring unit for the photo-optical measurement of blood coagulation reactions of blood plasma samples, and it also has a control unit, favorably a computer 37 (depicted schematically in FIG. 1), connected to these. The automatic apparatus is constructed in such a way that its test sample dispenser contains a module R moving the sample container racks 23 and a sampling arm MK. Its reagent dispenser has a reagent holder module 3 and a reagent-dispensing arm K2. Its further units have a modular construction, where the output of the cuvette-dispensing module C joins a system of storage tracks 31 connected to the nests of the rotatable disc of the incubation module 1. The cuvette removal location 1A of the incubation module 1, the reagent removal location 3A of the reagent holder module 3, the measuring location 2A of the measuring module 2 and the receptacle X where the cuvettes are collected after measuring are all arranged along the same circular arc.

In the geometric center of the circular arc there is the common axis of rotation 4 of the cuvette-moving arm K1 and the reagent-dispensing arm K2 moved in horizontal and vertical planes. The reagent-dispensing arm K2 has rinsing and washing positions W3 and W2. Furthermore, the sampling position 26 of the module R moving the sample container racks 23, one cuvette nest of the rotatable disc of the incubation module 1, the removal point EA of a further emergency module E, and further rinsing position W4 and washing position W1 of the sampling tip MK* are arranged along a further circular arc. The axis of rotation of the sampling arm MK is placed in the geometric center of the further circular arc.

According to FIG. 1, the sample container racks 23 containing sample tubes filled with test samples—are arranged on a movable tray 24 in fixed lines 24A open at least on one side. The sample container racks 23 can be moved horizontally via the movable tray 24, and one of the sample container racks 23, together with the sample tubes on it filled with test sample, may be positioned at the desired sampling position 26 by pushing it in and out from the movable tray 24—this tool is not shown separately in FIGS. 1-2.

FIG. 2 shows the top view of the automatic apparatus, and at the same time it convincingly demonstrates the space saving advantages of the modular construction and the arrangement of the cuvette-moving arm K1, the reagent-dispensing arm K2 and the sampling arm MK along a circular arc according to the invention. With cuvettes in the cuvette gap 19 the output of the cuvette-dispensing module C joins a system of storage tracks 31 connected to the receiver nests 33 of the rotatable disc 32 of the incubation module 1. The cuvettes are forwarded to the system of tracks 31 by a cuvette forwarding moving tool drive 21.

According to FIG. 2 the cuvette-moving arm K1 and the reagent-dispensing arm K2 are mounted on a common axis of rotation 4 placed in the geometric center O of a circular arc, and they may be rotated and moved in different directions along the given circular arc. The axis of rotation Z of the sampling arm MK is positioned in the center Y of a further circular arc. The top view clearly shows the area that can be used by moving the individual arms K1, K2, MK—along the circular arc and the further circular arc—and the preferred positions on it: the cuvette removal location 1A of the incubation module 1, the reagent removal location 3A of the reagent holder module 3, the measuring location 2A of the measuring module 2 and the receptacle X where the cuvettes are collected after measuring are all arranged along the same circular arc. The rinsing position W3 and washing position W2 of the reagent-dispensing arm K2 are also arranged along this circular arc. Furthermore, the sampling position 26 of the module R moving the sample container racks 23, one cuvette nest 33 of the rotatable disc 32 of the incubation module 1, the removal point EA of a further emergency module E, and a further rinsing position W4 and washing position W1 of the sampling tip MK* are arranged along a further circular arc. The flowcharts in FIGS. 3-8 make it easier to understand the harmonic and controlled operation of the individual modules C, 1, 2, 3, E, R and arms K1, K2, MK of the solution according to the invention, and at the same time they also illustrate the basic principles of the automatic operation of the control unit, favorably computer 37.

In the automatic apparatus according to the invention the harmonic and controlled operation of the individual modules (see FIGS. 1 & 2) is described in detail below. The harmonic and controlled operation of the modules is based on a reporting system (see FIGS. 3-8), in which the electric impulses indicating the individual functions (for example impulses generated by a change in light intensity) and/or data are converted into computer data series, and after processing these data series on the computer, the following instructions and steps determined in the computer program(s) (for example sampling, reagent-dispensing, cuvette-moving, carrying on rotating the rotatable disc by a step motor, etc.) are performed. It is all based on the work list set up on the control unit, favorably in computer program(s), for preparing and forwarding the samples selected for the tests according to the desired reaction(s).

In the automatic apparatus according to the invention the dispensing of the cuvettes is realized as described below. A plurality of cuvettes stored unarranged in the cuvette-dispensing module C are arranged at the bottom of a slight incline according to the principle of gravitation in the cuvette gap 19 of arranging elements that are parallel to each other or are situated at an angle with respect to each other.

The arranging elements that are parallel to each other or are situated at an angle with respect to each other, together with the cuvettes arranged between their edges, are lifted from the module C up until its output opening using mechanics driven by motor transmission. Through this output opening the cuvettes arranged side by side are removed preferably one by one, they are forwarded for further use with the help of a cuvette forwarding and moving device 21 driven along the edge of the lifted arranging elements, and the arranging elements that are parallel to each other or are situated at an angle with respect to each other are returned to their initial position at the bottom of the incline. When in the path of the cuvette forwarding and moving device 21 there are no more arranged cuvettes to be forwarded, the process is started again by lifting up the arranging elements from their initial position.

The cuvettes arriving from the cuvette-dispensing module C are forwarded into a system of tracks 31 for temporary storage constructed in the stationary part of the incubation module 1 kept at a permanent temperature set uniformly to suit the desired measurement. From this system of tracks 31 for temporary storage the cuvettes are forwarded into nests 33 of the rotatable disc 32 created in the moving part of the above incubation module 1 kept at a permanent temperature. The cuvette content of the rotatable disc 32 is checked at least in one position, favorably at least at the joining point of the system of tracks 31 for temporary storage and the rotatable disc 32, with the light emitting and light sensing elements situated along the nests 33 of the rotatable disc 32. The check is based on that in the lack of cuvettes the light emitting and light sensing elements situated along the nest 33 of the rotatable disc 32 report a change in light intensity to the control unit, favorably a computer, which issues a command to suspend sample dispensing.

For dispensing samples in the cuvettes the sample container racks 23 are arranged in the module R moving them on a movable tray moving in a given direction, favorably direction x, in open lines favorably fixed in direction y. The sample container racks 23 arranged in fixed lines, together with the movable tray 24, are moved forwards/backwards along an x axis favorably with the help of a horizontal step motor controlled by a control unit 37, favorably a computer, and by this one of the simultaneously moved sample container racks 23 selected as required is brought into a preferred position with the movable tray 24. In order to accomplish this, the sample container rack(s) 23 and the sample tubes receive an individual identifier (for example a serial number) from the control unit, favorably a computer, in its work list. With the help of position detecting and moving mechanics, the sample container rack 23 brought into a preferred position according to the work list is pushed along a y axis from the movable tray 24 towards the sampling position 26 and moved forwards/backwards along the y axis. As a result of this, in accordance with the control command, the sample tube(s) according to the work list is (are) forwarded to the sampling position 26 of the automated analyzer, and after sampling the sample container rack 23 is returned from the sampling position 26 onto the movable tray 24.

For dispensing samples into the cuvettes, the sampling arm MK situated near the incubation module 1 is assigned selected positions, which are the following: sampling position, sample dispensing position, sampling tip rinsing position, sampling tip washing position. On the instructions of the control unit 37, favorably a computer, the sampling arm MK moves into a sampling position, when, following an instruction again, the sample tube(s) according to the work list mentioned above is (are) forwarded to the sampling position 26 of the automatic analyzer. In order to avoid collisions the sampling arm MK, in its sampling position, blocks the movement of the sample container rack 23. Following a further control command the sampling arm MK turns into a sample dispensing position, when in the incubation module 1 kept at a permanent temperature set uniformly to suit the desired measurement the rotatable disc 32 containing the cuvettes is brought into a preferred position, which is the sample dispensing position.

The automatic apparatus according to the invention also contains an emergency module E. The emergency module E has a disc driven by motor transmission, and on the emergency disc the removal point is placed on a further circular arc as the sampling arm MK rotates. Favorably, on the emergency disc places are created for accommodating sample tubes, tubes containing washing liquid. Emergency samples, control samples and calibration samples are placed in the sample tubes. The sample tubes placed on the emergency disc are individually tracked by control, with the help of barcodes or serial numbers. Following the control commands, the sampling arm MK takes out the desired amount from the emergency sample or control sample or calibration sample rotated to the removal point of the emergency disc, into the cuvette waiting in the sample dispensing position of the rotating disc 32 of the incubation module 1.

In the incubation module, the cuvettes containing test samples are tracked with the help of a control program individually, with the help of a barcode or serial number. In the incubation module 1, in a different preferred position, in the reagent-dispensing position of the rotatable disc 32 rotated with the help of a program-controlled step motor, reaction mixture is created by dispensing a required amount of reagent(s) into the cuvettes filled with test samples.

Favorably, reagents are dispensed with the reagent-dispensing arm K2 mounted on a common axis of rotation 4 with the cuvette-moving arm K1. The reagent-dispensing arm K2 is also given preferred positions, which are the following: reagent holder module 3 reagent removal point 3A, measuring module 2 measuring points 2A, incubation module 1 cuvette removal point 1A, and reagent-dispensing arm K2 washing position W2. The preferred positions listed above follow the favorable modular arrangement of the automatic apparatus according to the invention along circular arc(s) as described above.

The reagent holder module 3 contains a rotatable disc with a position sensor, and on this disc the reagent holder vessels/tubes are situated along concentric circles. The head of the reagent-dispensing arm K2 designed to perform suction and discharge functions sucks up the required amount of reagent at the reagent removal point 3A of the reagent holder module 3. Controlled reagent-dispensing by the reagent-dispensing arm K2 takes place in the cuvettes in the reagent-dispensing position of the rotatable disc 32 of the incubation module 1 and in the cuvette(s) containing a test sample moved to the measuring point(s) 2A of the measuring module 2.

In the incubation module 1 the cuvettes containing a test sample or reaction mixture may be kept at a permanent temperature suiting the desired reaction, for individual incubation period(s) suiting their content and controlled with a control unit, favourably a computer. After the individual incubation period(s) has (have) expired, the individual incubated cuvettes are rotated by the control unit to the cuvette removal point 1A of the rotatable disc 32, from where they are moved to the measuring point(s) 2A of the bioanalytical automatic apparatus with the help of the cuvette-moving arm K1 mounted on a common axis of rotation 4 with the reagent-dispensing arm K2 and constructed in such a way that it supports the edge of the cuvette. The controlled cuvette-moving arm K1 is also given selected positions, which are the following: incubation module 1 cuvette removal point 1A, measuring module 2 measuring points 2A, receptacle X where cuvettes are collected after measuring. The preferred positions listed above follow the favorable modular arrangement of the automatic apparatus according to the invention along circular arc(s) as described above.

A favorable arrangement of eight measuring locations 2A in the measuring module 2 ensures the continuous operation of the reagent-dispensing arm K2 as required, undisturbed by the movement or position of the cuvette-moving arm K1 between the measuring points 2A.

In a favorable form of execution of the automatic apparatus, optical measuring locations 2A are created in the measuring module 2, where turbidimetric and nephelometric light intensity characteristic of the tested reaction is detected at the same place and at the same time, in optical measuring cells having a small space demand. Most favorably it is solved by placing light emitting diodes in the measuring cells and emitting light of two-three wavelengths simultaneously onto the sample. In turbidimetric measurements (for example D-dimer reaction indicating fibrin degradation) the light passing through the sample is detected in the optical measuring cells. In nephelometric measurements (for example PT prothrombin time, APTT activated partial thromboplastin time, etc.) the scattered on sample light of two light sources of the same wavelength or of different wavelengths positioned at right angles with respect to each other is detected with a detector in the optical measuring cells. The results of measuring the light passing through and scattered on the sample are forwarded to the signal processing system, from where they are forwarded as a computer data series to the control unit 37, preferably a computer, and evaluated.

An exemplary solution according to the present disclosure has a space-saving construction, is fast in operation and is user-friendly by being easy to handle, has an integrated construction, that, makes it unnecessary to use additional moving structures during its operation, in practice.

Finally, it should be noted that the term “comprising” does not exclude other elements or features, and that use of the terms “a” or “an” does not necessarily exclude a plurality, in the sense that singular reference of an element does not exclude the plural reference of such elements. The verb ‘comprise’ and its conjugations do not exclude the presence of elements or steps other than those listed in any claim or the specification as a whole. The mere fact that certain measures are recited in mutually different dependent claims does not indicate that a combination of these measures cannot be used to advantage. Furthermore, elements described in association with different versions may be combined. Finally, it should be noted that the above-mentioned examples, and versions illustrate rather than limit the invention, and that those skilled in the art will be capable of designing many alternative implementations without departing from the scope of the invention as defined by the appended claims. As equivalent elements may be substituted for elements employed in claimed invention to obtain substantially the same results in substantially the same way, the scope of the present invention is defined by the appended claims, including known equivalents and unforeseeable equivalents at the time of filing of this application. Thus, in closing, it should be noted that the invention is not limited to the abovementioned versions and exemplary working examples. Further developments, modifications and combinations are also within the scope of the appended patent claims, and are placed in the possession of the person skilled in the art from the present disclosure. Accordingly, the techniques and structures described and illustrated previously herein should be understood to be illustrative and exemplary, and not necessarily limiting upon the scope.

What is claimed is:

1. A process for diagnostic testing comprising the steps of:
maintaining empty cuvettes under controlled temperature;
arranging test samples in sample tubes arranged in sample container racks;
transferring test samples to test cuvettes;
incubating the test cuvettes;
measuring sample reactions of the test cuvettes photo-optically;
providing an incubated cuvette removal location;
providing a test reagent removal location;
providing at least one test sample measuring location;
arranging the incubated cuvette removal location, the test reagent removal location, and the at least one test sample removal location along a first circular arc with a geometric center;
on the geometric center locating a common axis of rotation for at least one cuvette-moving arm and at least one reagent-dispensing arm;
providing rinsing and washing positions for the at least one reagent-dispensing arm; and,
providing a receptacle for removal of test cuvettes after measurement.

2. A process for diagnostic testing as claimed in claim 1, further comprising the steps of:
providing a sampling position, a sample dispensing position, an additional rinsing position, and an additional washing position, all on a second circular arc with a second geometric center;
on the second geometric center locating a second axis of rotation for a sampling arm; moving the at least one cuvette-moving arm and the at least one reagent-dispensing arm along the first horizontal arc in horizontal and vertical planes; and,
moving the sampling arm along the second horizontal arc in horizontal and vertical planes.

3. A process for diagnostic testing as claimed in claim 2, further comprising the step of:
providing an additional position along the second circular arc to serve as an emergency disc removal point.

4. A process for diagnostic testing as claimed in claim 1, further comprising the step of:
moving the cuvette-moving arm and the reagent-dispensing arm in different planes.

5. A process for diagnostic testing as claimed in claim 4, further comprising the step of:
locking movement of the reagent-dispensing arm within a vertical plane by the movement of the cuvette-dispensing arm in a horizontal plane.

6. A process for diagnostic testing as claimed in claim 5, further comprising the step of:
locking movement of the sample container rack when the sampling arm is in a sampling position.

7. A process for diagnostic testing as claimed in claim 2, further comprising the step of:
locking movement of the emergency disc when the sampling arm is in a sample dispensing position.

8. A process for diagnostic testing as claimed in claim 7, further comprising the step of:
locking movement of the incubation disc in sample dispensing position when the sampling arm is in sample dispensing position.

9. A process for diagnostic testing as claimed in claim 1, further comprising the step of:
locking movement of the incubation disc in reagent-dispensing position when the reagent-dispensing arm is in reagent dispensing position.

10. A process for diagnostic testing as claimed in claim 7, further comprising the step of:
locking movement of the reagent holder disc in reagent removal position when the reagent-dispensing arm is in reagent dispensing position.

11. A process for diagnostic testing as claimed in claim 1, further comprising the step of:
situating the rinsing and washing positions for the at least one reagent-dispensing arm along said first circular arc.

12. Apparatus for automated diagnostic testing comprising:
a cuvette dispensing unit;
a test sample dispenser;
a reagent dispenser;
an incubation unit;
a measuring unit configured to make photo-optical measurements;
a control unit in operative communication with said cuvette dispensing unit, said test sample dispenser, said reagent dispenser, said incubation unit, and said measuring unit;
said test sample dispenser containing a sample rack moving module, said test sample dispenser having a rotatable sampling arm, said sampling arm having a sampling tip, said sample rack moving module having a sampling position;
said reagent dispenser having a reagent holder module, said reagent dispenser having a reagent-dispensing arm;
said cuvette dispensing unit having a cuvette output, said cuvette output being connected to at least one storage track, said storage track being connected to supply receivers of said incubation unit, said receivers being located in a rotatable disc;
a cuvette removal location in said incubation unit;
a reagent removal location in said reagent holder module;
at least one measuring location in said measuring unit;
a collection receptacle configured to receive used cuvettes;
said cuvette removal location, said reagent removal location, said at least one measuring location, and said collection receptacle all being situated along a first common circular arc having a first geometric center;
a cuvette-moving arm configured to move in horizontal and vertical planes, and a reagent-dispensing arm configured to move in horizontal and vertical planes, said cuvette-moving arm and said reagent-dispensing arm having a common axis of rotation passing through said first geometric center;
said reagent-dispensing arm having a rinsing position, and reagent-dispensing arm having a washing position;
an emergency module, said emergency module having a removal location;
a rinsing position for said sampling tip, and a washing position for said sampling tip;
said removal location, said rinsing position and said washing position for said sampling tip, and one of said receivers of said rotatable disc all being situated along a second circular arc having a second geometric center; and,
said sampling arm having an axis of rotation passing through said second geometric center of said second circular arc.

13. The apparatus for automated diagnostic testing as claimed in claim 12, wherein:
motions of said cuvette-moving arm, said reagent-dispensing arm, and said sampling arm are all controlled by said control unit.

14. Apparatus for automated diagnostic testing as claimed in claim 12, further comprising:
a plurality of measuring locations in said measuring unit, said plurality of measuring locations being situated along said first common circular arc; and,
said cuvette-moving arm having a position reaching to said incubation unit, and said reagent-dispensing arm having a position reaching to said reagent holder module.

15. Apparatus for automated diagnostic testing as claimed in claim 12, further comprising:
said sampling arm having a first position reaching to sample rack moving module, said sampling arm having a second position reaching to said incubation unit, and said sampling arm having a third position reaching to said emergency module.

16. Apparatus for automated diagnostic testing as claimed in claim 12, further comprising:
said reagent-dispensing arm rinsing position and washing position are situated on said first common circular arc; and,
said sampling tip rinsing position and washing position are situated on said second circular arc.
